(19) United States
(12) Patent Application Publication
(10) Pub. No.: US 2014/0309570 A1
(43) Pub. Date: Oct. 16, 2014

(54) Chest Brace

(71) Applicant: ALFRED HEALTH, Melbourne (AU)

(72) Inventor: Louise Mary FULLER, Balwyn (AU)

(73) Assignee: ALFRED HEALTH, Melbourne (AU)

(21) Appl. No.: 13/860,245

(22) Filed: Apr. 10, 2013

(51) Int. Cl. A61F 5/02

(52) U.S. Cl. CPC A61F 5/02
USPC 602/19

(57) ABSTRACT

A chest brace comprising a mouldable thermoplastics pressure plate with an adjustable waist strap and adjustable cross over shoulder straps whereby the straps are adjusted so that the plate transmits an antero-posterior force to the chest of the patient.

### INTRODUCTION

This invention relates to a chest brace particularly for use by lung transplant patients.

### BACKGROUND OF THE INVENTION

Bilateral anterior thoracotomies and a transverse sternotomoy are used with a “clam shell” incision to allow for the replacement of lungs sequentially. A clam shell incision is associated with division or “cracking” of the sternum. It has been recognised that patients undergoing this operation can suffer from significant post operative pain caused by bone overriding and disruptions at the sternum site. The movement caused by breathing, coughing, sneezing etc can cause sternum instability which can have serious end results. It is the issues associated with the sternum instability following bilateral sequential lung transplants that has brought about the present invention.

### SUMMARY OF THE INVENTION

According to the present invention there is provided a chest brace comprising a mouldable thermoplastic pressure plate with an adjustable waist strap and adjustable cross over shoulder straps whereby the straps are adjusted so that the plate transmits an anteroposterior force to the chest of the patient.

The pressure plate is preferably trimmed and moulded to the chest shape of the patient. The straps are preferably inextensible and adjustable through use of hook and eye fasteners. In a preferred embodiment adjustable shoulder strap links join the front of the pressure plate to a ring located adjacent the spine of the patient and diagonal shoulder straps extend down from the ring to the adjustable waist strap.

In a preferred embodiment the thermoplastics material is a conventional thermoplastics used in the orthotics industry whereby the material becomes malleable and mouldable when heated and assumes a rigid configuration at room temperature.

### DESCRIPTION OF THE DRAWINGS

An embodiment of the present invention will now be described by way of example only with reference to the accompanying drawings in which:

FIG. 1 is a front view of a male patient wearing a chest brace in accordance with an embodiment of the invention,

FIG. 2 is a rear view of the patient wearing the brace, and

FIG. 3 is a front perspective view of the patient wearing the brace.

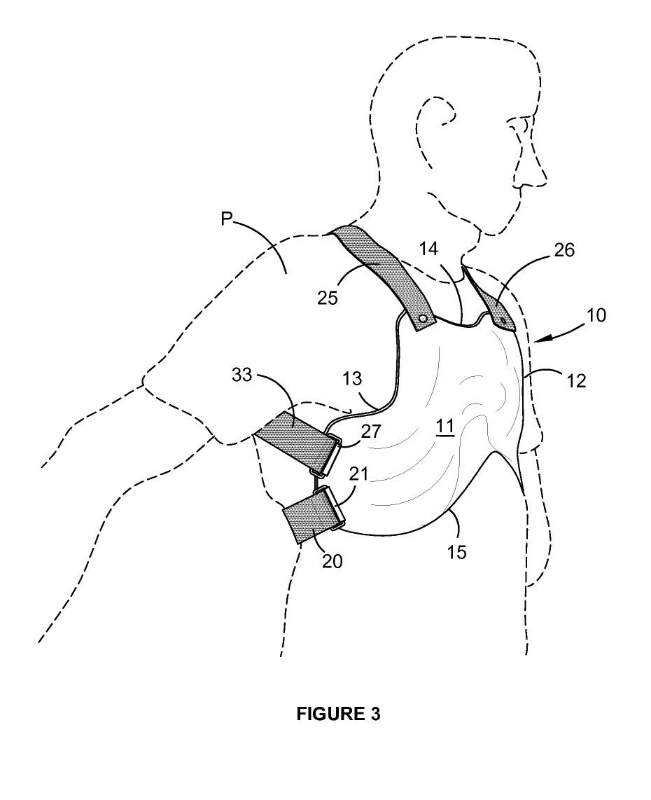

### DESCRIPTION OF AN EMBODIMENT

A transverse sternotomy with bilateral anterior thoracotomies require what is known as a clam shell incision that extends transversely across the chest of the patient. This incision is used for bilateral sequential lung transplantation and aortic aneurysm repair. The clam shell incision is a horizontal cut at a level between the fourth and fifth rib. The cut allows for optimal access to mediastinun, aorta and pleural cavities. However, patients that undergo bilateral sequential lung transplantation with a transverse sternotomy often suffer post operative pain caused by bone overriding deformities as the bone endeavours to heal. Coughing, sneezing or even breathing can cause bone movement that can result in wires that hold bone ends together breaking and severe displacement of the bone causing increased pain and infection.

The chest brace 10 illustrated in the accompanying drawings has been specifically designed so that it is worn post operatively by patients who have undergone a clam shell incision for at least 6 weeks after the operation during non-sleeping and non-showering hours. The chest brace 10 essentially comprises a mouldable pressure plate 11 of thermoplastics specifically shaped to suit the patient P. Inextensible but adjustable straps 20, 25, 26 are used to secure the plate 11 to the chest of the patient so that the plate transmits an anteroposterior (front to back) force to the chest of the patient P.

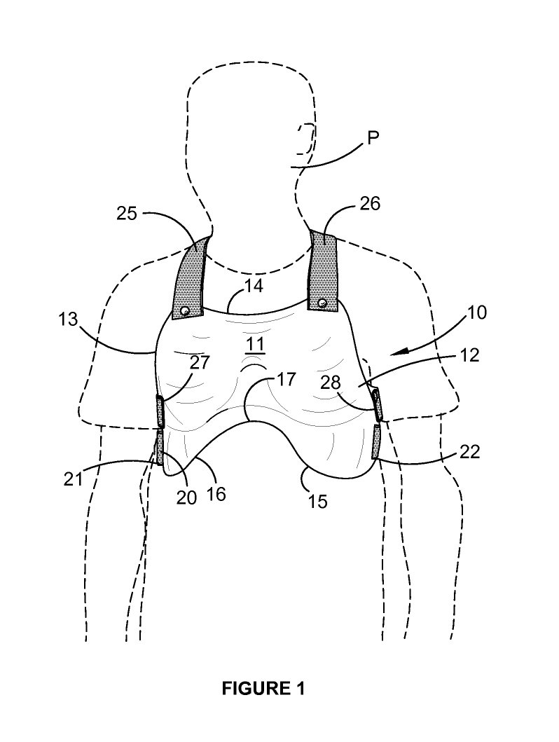

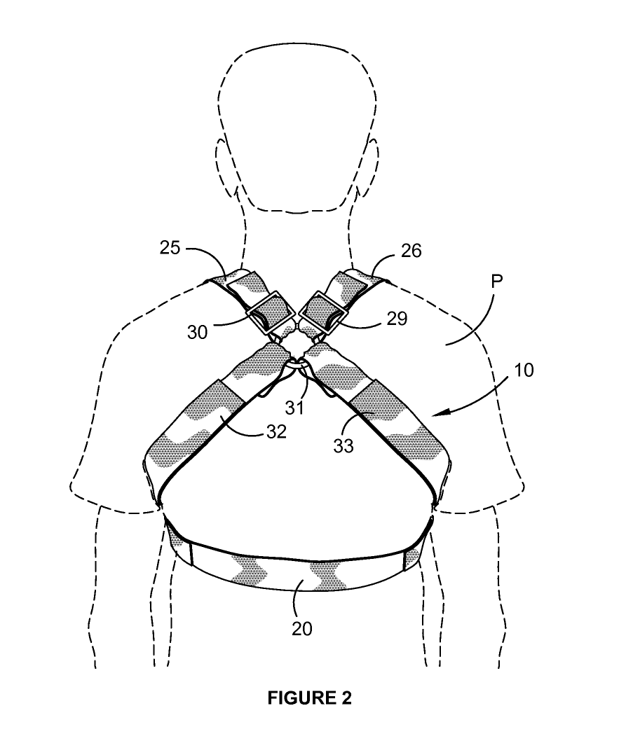

As shown in the accompanying drawings, the plate 11 is profiled or curved to spread evenly across the chest of the patient. As shown in FIG. 1, when viewed two dimensionally from the front, the plate 11 is loosely rectangular with side edges 12 and 13, a top edge 14 and a lower edge 15. The lower edge has a U-shaped cut out 16 with the pit 17 of the cut out 16 located at the centre of the chest cavity. A waist strap 20 with adjustable points 21, 22 at the base of each side edge 12, 13 of the plate 11 extends substantially horizontally across the back of the patent P as shown in FIG. 2. At each corner of the upper edge 14 left and right shoulder straps 25, 26 are attached to the plate 11 to extend over the shoulders of the patient and then diagonally across the back to join the plate 11 at left and right attachment points 27, 28 on the side edges 12, 13 of the plate above the waist strap 20 attachment positions 21, 22. The shoulder straps 25, 26 split into two links namely, upper links 29, 30 that are attached to a plastics ring 31 and lower links 32, 33 that extend from the ring 31 down and around the sides of the patient to join the attachment points 27, 28 on the plate 11. The ring 31 locates and evenly distributes the forces applied by the straps 25, 26.

The straps are made of inextensible fabric, webbing or nylon that are adjustable by use of hook and eye fasteners commonly sold under the trade mark VELCRO. Each link 29, 30, 32, 33 of the shoulder straps 25, 26 can be appropriately tensioned as can the waist strap 20 to ensure that the plate 11 provides an adequate force extending across the chest from the front to the back of the patient P.

The plate 11 is manufactured from thermoplastics material that is generally used in the orthotics industry. The material is easy to cut and when heated with a heat gun becomes very malleable which allows the plate 11 to be cut and shaped to the chest of the patient P. When the plate 11 is at room temperature it assumes a fairly rigid configuration to provide the required forces on the chest of the patient.

It is understood that the plate 11 may be appropriately perforated and may be lined with a suitable backing material. The plate 11 can also have specific apertures cut into it to provide access to wounds and entry for drainage, tubes etc.

These holes can be filled when not required. In cutting the plate to suit the patient key areas to ensure exact fitment are under the arms and ensuring that the lower edge 15 of the plate 11 is under the last rib. The upper edge 14 can also be configured to suit the neck and shoulder structure of the patient. Although the preferred embodiment shows the brace 10 on a male patient it is understood that this brace is equally applicable to women. The three dimensional curvature of the plate 11 would be adjusted to accommodate the breasts of the female patient.

The chest brace 10 described above is light and thin yet able to closely conform to the chest of the particular wearer so that once the straps are appropriately tensioned an even force is applied from the front to the back of the wearer. This force substantially reduces the likelihood of bone displacement as the wearer moves about during daylight hours. It is envisaged that a patient would wear the brace for at least six weeks after the operation and possibly longer, and testing has shown that the incidents of acute pain and bone displacement in patients wearing a brace of this kind is substantially reduced.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A chest brace comprising a mouldable thermoplastics pressure plate with an adjustable waist strap and adjustable cross over shoulder straps whereby the straps are adjusted so that the plate transmits an antero-posterior force to the chest of the patient.

2. The chest brace according to claim 1 wherein the plate is trimmed and moulded to the chest shape of the patient.

3. The chest brace according to claim 1 wherein the plate is planar with front and rear surfaces which conform to the chest of the patient and define an upper edge, opposed side edges and a base edge.

4. The chest brace according to claim 3 wherein the cross over shoulder straps join the upper edge of the front of the pressure plate to a ring located adjacent the spine of the patient and from the ring down to the side edges near the base edge.

5. The chest brace according to claim 4 wherein each shoulder strap comprises an upper link extending from an upper corner of the plate over the shoulder of the patient to the ring and a lower link extending from the ring to mounting points on the side edges of the plate near the base edge.

6. The chest brace according to claim 3 wherein the waist strap extends from mounting points on the side edges near the base edge across the back of the patient.

7. The chest brace according to claim 1 wherein the straps are inextensible but length adjustable.

8. The chest brace according to claim 1 wherein the thermoplastic plate is malleable and mouldable when heated but assumes a substantially rigid configuration at room temperature.
